(19) United States
(12) Patent Application Publication
(10) Pub. No.: US 2021/0252189 A1
(43) Pub. Date: Aug. 19, 2021

(54) CELLULOSE NANOFIBER BIOMATERIAL

(71) Applicants: Alliance for Sustainable Energy, LLC, Golden, CO (US); New Vision BioLOGIC, Inc., Aurora, CO (US)

(72) Inventors: Stephen R. DECKER, Berthoud, CO (US); Peter N. CIESIELSKI, Arvada, CO (US); Chad J. RONHOLDT, Aurora, CO (US)

(21) Appl. No.: 17/269,422

(22) PCT Filed: Aug. 20, 2019
(86) PCT No.: PCT/US19/47264

§ 371 Date: Feb. 18, 2021

Related U.S. Application Data
(60) Provisional application No. 62/719,779, filed on Aug. 20, 2018.

(51) Int. Cl. A61L 27/20; A61L 27/38; A61L 27/54; A61L 27/36

(52) U.S. Cl. CPC A61L 27/20; A61L 27/3847; A61L 27/54; A61L 27/3608; A61L 2300/428; A61L 2430/02; A61L 2300/102; A61L 2300/406; A61L 2300/414; A61L 2400/12

(57) ABSTRACT

Formulations of cellulose nanofiber useful for osteoinduction are provided.

### CROSS-REFERENCE TO RELATED APPLICATION

This application claims priority under 35 U.S.C. § 371 to PCT application no. PCT/US2019/047264 with an international filing date of 20 Aug. 2019 which claims priority under 35 U.S.C. § 119 to U.S. Provisional Patent Application No. 62/719,779 filed on 20 Aug. 2018, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### SUMMARY

In an aspect, a formulation capable of inducing bone growth comprising cellulose nanofiber is disclosed. In an embodiment, the formulation contains demineralized bone matrix. In another embodiment, the formulation contains alginate. In yet another embodiment, the formulation contains cellulose nanofiber and demineralized bone matrix. In an embodiment, the formulation contains cellulose nanofiber and alginate. In another embodiment, the formulation contains cellulose nanofiber, demineralized bone matrix and alginate.

In an aspect, a method for inducing the growth of bone in a subject is disclosed that includes delivering a formulation of cellulose nanofiber to the subject.

In another aspect, a method for inducing the growth of bone in a subject is disclosed that includes delivering a formulation of cellulose nanofiber and demineralized bone matrix to the subject.

In yet another aspect, a method for inducing the growth of bone in a subject is disclosed that includes delivering a formulation of cellulose nanofiber, demineralized bone matrix and alginate to the subject.

In an aspect disclosed herein is a bone repair composition comprising cellulose nanofiber useful as an osteoconductive matrix for cells to attach and adhere to during osteogenesis; and a derived inductive component, comprising allogenic cells and tissues added to stimulate and promote osteogenesis; and further comprising autologous derived cells and tissues; and further comprising recombinant proteins, cytokines, and growth factors; and further comprising compounds useful for bone remodeling comprising calcium, magnesium, and vitamin D; and further comprising compounds useful for the mitigation of infection comprising antibiotics and heavy metal ions. In an embodiment the composition is biocompatible, non-toxic, anti-inflammatory, non-immunogenic biomaterial derived from renewable sources. In an embodiment, the composition is derived from wood, plants, trees, paper pulp, bacteria, and algae. In an embodiment, the composition comprises cellulose nanofiber derived from plant-based material with a length of between 50 nanometers and 100 micrometers and diameter of 2 nanometers and 20 nanometers. In an embodiment, the composition further comprising cellulose nanofibers comprising fiber bundles with lengths of between about 50 nanometers and 1 millimeter and diameters of between about 2 nanometers and 3 micrometers. In an embodiment, the composition comprises fiber bundles have a water content of between about 50% to about 80%. In an embodiment, the composition comprises fiber bundles with an alginate concentration of between about 1% to about 10% by weight. In an embodiment, the composition further comprising fiber bundles and powder cut from cortical bone demineralized to a calcium content of no more than 10%. In an embodiment, the composition further comprising fiber bundles and bone chips and/or bone cubes cut from non-demineralized cancellous bone with sizes of between about 1 mm to about 15 mm. In an embodiment, the composition further comprises calcium chloride at a concentration from about 0.1 M to about 10 M wherein the solution is allowed to cross-link between about 0.1 min to about 60 min. In an embodiment, the composition further comprises less than 10% water by weight, less than 50% water by weight, less than 80% water by weight and greater than 80% water by weight. In an embodiment, the composition further comprises an excipient selected from the group consisting essentially of alginate, glycerol, lecithin, sodium carboxy methyl cellulose, hyaluronic acid and derivatized hyaluronic acid. In an embodiment, the composition further comprises a cross-linking agent selected from multivalent cations comprising calcium, magnesium, and further comprising counter ions including chlorides, sulfates, carbonates, and nitrates. In an embodiment, the composition further comprises vitamin A, vitamin B, vitamin C, vitamin D, vitamin E and vitamin K. In an embodiment, the composition further comprises a demineralized bone matrix component. In an embodiment, the composition further comprises a non-demineralized bone matrix component. In an embodiment, the composition further comprises growth factors, cytokines, antimicrobial agents, antifungal agents, bioglass and its derivatives, tri-calcium phosphates and its derivatives, chitosan and its derivatives, collagen and its derivatives.

In an aspect, disclosed is a method for repairing bone using the composition of claim 13 wherein the cross-linking agent is added to the rest of the composition and wherein the reaction time of the cross-linking agent with the rest of the composition is directly proportional to resorption time in-vivo. In an embodiment, the method includes using a composition wherein the concentration of the cross-linking agent is proportional to resorption time in-vivo. In an embodiment, the method includes a step wherein the resorption time in-vivo is between 1 month to about 12 months.

Other objects, advantages, and novel features of the present invention will become apparent from the detailed description of the invention when considered in conjunction with the accompanying drawings and claims.

### BRIEF DESCRIPTION OF THE DRAWINGS

None

### DETAILED DESCRIPTION

Disclosed herein are cellulose nanofibers (CNF) that are a novel biomass-derived material for biomedical applications. The CNF disclosed herein possess highly tunable physical properties (porosity, viscosity, etc.), are non-toxic, non-immunogenic and have the ability to integrate calcium needed for bone growth. Disclosed herein are formulations of CNF without demineralized bone matrix (DBM) that perform as well as biomaterials with DBM, the lack of DBM provides a significant reduction in cost to manufacture and use the CNF materials disclosed herein. Further, formulated CNF with calcium chloride and alginate have demonstrated physical properties that are compelling to the surgical community while promoting osteocyte activity.

Also disclosed herein are methods to determine if CNF possesses osteoinductive or osteoconductive properties as a carrier material alone. In an embodiment, CNF does not present a biocompatibility problem in an in-vivo model. In another embodiment, a CNF+DBM product results in osteoinductivity within the in-vivo model.

Disclosed herein are methods of use and compositions of CNF to evaluate the inductive, conductive and biocompatibility characteristics of CNF as a biomaterial for use in biomedical applications such as, for example, spinal fusion and general orthopedic use.

In an embodiment CNF is a material composed of nanosized cellulose fibrils with a high aspect ratio (length to width ratio). In an embodiment, fibril widths are from about 5 to about 20 nanometers with a wide range of lengths, typically several micrometers. In an embodiment, wherein the length of the CNF is from about 50 nanometers to 1 millimeter and the diameter of the CNF is from about 2 nanometers to 1 micrometer. In an embodiment, CNF is pseudo-plastic and exhibits thixotropy, the property of certain gels or fluids that are thick (viscous) under normal conditions but become less viscous when shaken or agitated. In an embodiment, when the shearing forces are removed a CNF gel, it regains much of its original state. In an embodiment, CNF are isolated from any cellulose containing source including wood-based fibers (pulp fibers) through high-pressure, high temperature and high velocity impact homogenization, grinding or microfluidization. In an embodiment, the CNF used herein is derived and/or isolated from a renewable source. In an embodiment, CNF is obtained from native fibers by an acid hydrolysis, resulting in highly crystalline and rigid nanoparticles which are shorter (100s to 1000 nanometers) than the nanofibrils obtained through homogenization, microfluiodization or grinding routes.

In an embodiment DBM is allograft bone that has had the inorganic mineral removed, leaving behind an organic collagen matrix. In an embodiment, DBM is processed and terminally sterilized prior to implantation to remove the risk of disease transmission or an immunological response. This processing removes the osteogenic and osteoinductive properties of the graft, leaving only an osteoconductive scaffold. In an embodiment, these scaffolds are available in a range of preparations (such as morselized particles and struts) for different orthopaedic applications. In an embodiment, and without being limited by theory, DBM has superior biological properties to undemineralised allograft bone, as the removal of the mineral increases the osteoinductivity of the graft.

Raw CNF is almost 97% water and 3% CNF and therefore it was necessary to significantly reduce the water content so that the raw CNF was easier to handle (i.e. more putty like). This water removal step was done manually by pressing the CNF between a hydrophobic filtration cloth (Miracloth) to concentrate the CNF into a pancake-batter-like consistency.

Once the CNF was at a target dry matter weight of about 25%, it was set aside while the DBM powder was prepared. The powder samples received from ABT had larger lattice-like chunks dispersed throughout the individual packages. Previous formulation work with the same DBM lot, noted that these larger DBM clumps (greater than about 300 μm) had a difficult time dispersing and mixing into the final putty/flowable gel forms. In addition, once prepared in the final formulation, these larger chunks served as fracture points where the implant would crumble around these larger particles and not hold its form when compressed or expelled through a syringe. Therefore, all samples were further crushed using a mortar pestle to a consistent fine power on the lower end of the particulate scale (about 125 μm). This pestle step was performed while all DBM was still in the product Tyvek packaging to minimize waste and maintain aseptic conditions since the pestle was not sterile. This fine powder was added to varying concentrations of alginate, which was used as a binder and cross-linking agent to lock the layers of DBM within the implant. Previous formulation experimentation also suggested that the DBM should be added to the alginate mixture first as it was much easier to achieve a very homogenous distribution of DBM throughout the alginate as compared to adding it to the CNF then adding the alginate. The latter formulation becomes very dry as the hygroscopic DBM absorbs any remaining water in the CNF/alginate mixture and as such it becomes very difficult to mix all of the elements consistently without adding more water. Once thoroughly mixed, the DBM/alginate component was added to varying concentrations of raw CNF, see Table 1.

TABLE 1
Sample Preparation Matrix

| | NREL | NaAlg | dH₂O | CNF | DBM | total | Final | Final | Final |
|---|---|---|---|---|---|---|---|---|---|
| Sample | Sample | (mL) | (mL) | (g) | (g) | mass | NaAlg | CNF | DBM |
| ID | ID | 6.50% | 100% | 25% | 100% | (g) | (%) | (%) | (%) |
| RI-001/002—DBM | DBM(−) | 0.0 | 0.0 | 0.0 | 5.0 | 5 | 0.00 | 0.00 | 0.00 |
| Neg Control | | | | | | | | | |
| RI-003/004 CNF-F1 | 1C | 1.3 | 2.5 | 1.2 | 0 | 5 | 1.69 | 6.00 | 0.00 |
| CONTROL | | | | | | | | | |
| RI-005/006 CNF-F1 | 1 | 1.3 | 2 | 1.2 | 0.5 | 5 | 1.69 | 6.00 | 10.00 |
| ACTIVE | | | | | | | | | |
| RI-007/008 CNF-F2 | 2C | 2.3 | 1.5 | 1.2 | 0 | 5 | 2.99 | 6.00 | 0.00 |
| CONTROL | | | | | | | | | |
| RI-009/010 CNF-F2 | 2 | 2.3 | 0 | 1.2 | 1.5 | 5 | 2.99 | 6.00 | 30.00 |
| ACTIVE | | | | | | | | | |
| RI-011/012—DBM | DBM(+) | 0.0 | 0.0 | 0.0 | 5 | 5 | 0.00 | 0.00 | 100.00 |
| Pos Control | | | | | | | | | |
| RI-013/014 CNF-F3 | 3C | 3.8 | 0.0 | 1.2 | 0 | 5 | 4.94 | 6.00 | 0.00 |
| CONTROL | | | | | | | | | |
| RI-015/016 CNF-F3 | 3 | 3.8 | 0.0 | 1.2 | 1.5 | 6.5 | 3.80 | 4.62 | 23.08 |
| ACTIVE | | | | | | | | | |

In an embodiment, these different alginate and DBM concentrations comprised two test variables, which were reflected in the three different formulations tested.

In an embodiment, general observations of this formulation process were that as the alginate/DBM concentration increased, it required more vigorous mixing to fully hydrate the CNF to create a smooth consistency without leaving non-hydrated chunks of CNF in the mixture. These non-hydrated chunks compromised the flowability of the final mixture and created fracture points when manipulating the biomaterial. This step took 15 to 45 minutes to complete as higher concentrations of alginate/DBM were prepared.

The final step was to use a flash calcium chloride soak of about 30 seconds to cross-link the alginate. This was performed to provide the desired handling characteristics and to lock the DBM into place within the putty/gel matrix. It is also thought that this step would provide additional benefit from a cellular signaling perspective as calcium chloride is an essential element for osteogenesis. The calcium chloride step may also facilitate visibility of the implant on x-rays during the remodeling phase. General observations of this flash soak step were that the implant materials did not seem to change and therefore, the final formulation (CNF-F3) was soaked for a much longer period (about 2 min) in an effort to see if the implant became saturated with the calcium chloride. Following the 2 min soak, the implant material had a very crusty and hard outer layer with a spongy, soft center suggesting that there is a saturation gradient present. The hard, outer layer initially diminished the pliability of the biomaterials until the outer layer was mixed with the inner layers with subsequent compression, resulting in a firmer feel than previous formulations (CNF-F1 and F2). Since the toxicity and osteoinductive characteristics of CNF were investigated with experiments disclosed herein, the handling characteristics were not considered as an acceptance criterion. Thus, the calcium chloride step has a pronounced effect on the handling characteristics of the bioimplant.

The DBM negative controls consisted of guanidine inactivated DBM powder. Approximately 5 grams of DBM powder was placed in a 50 ml centrifuge tube with about 30 ml of 100 mM guanidine solution. The tube was vigorously mixed, heated to greater than about 40° C. then allowed to sit for 45 min on an orbital shaker to deactivate the bone morphogenic proteins.

Once all bulk samples were prepared, a total of sixteen sterile 50 ml conical tubes were labeled with a blinded sample identified and referenced in Table 2. A total of three different formulations were evaluated (CNF Formulation 1, 2 and 3 in addition to, one positive control and one negative control). All formulations and controls were tested in duplicate and each tube received 250±50 mg of sample for implantation.

All samples were prepared in a non-sterile environment with non-sterile utensils and therefore all samples required terminal sterilization prior to implantation. The samples were delivered for low-dose electron beam (E-beam) gamma sterilization.

All samples were packed into a carrier without dry ice and terminally irradiated using a dose range of 14.0-15.9 kGy and shipped directly to WuXi AppTec for implantation.

TABLE 2
Sample Key

| | WuXI Apptec | # | Rat ID |
|---|---|---|---|
| Test Article | BLINDED Sample ID | implants | (# of Rats) |
| DBM NEGATIVE | 001 | 1 | R-1 (1) |
| Control | 002 | 1 | |
| NCF-F1- | 003 | 1 | R-2 (1) |
| Control | 004 | 1 | |
| NCF-F1-DBM- | 005 | 1 | R-3 (1) |
| 10% | 006 | 1 | |
| NCF-F2- | 007 | 1 | R-4 (1) |
| Control | 008 | 1 | |
| NCF-F2-DBM- | 009 | 1 | R-5 (1) |
| 30% | 010 | 1 | |
| DBM POSITIVE | 011 | 1 | R-6(1) |
| Control | 012 | 1 | |
| NCF-F3 Control | 013 | 1 | R-7 (1) |
| | 014 | 1 | |
| NCF-F3-DBM- | 015 | 1 | R-8 (1) |
| 23% | 016 | 1 | |
| TOTALS | | 16 | 8 |

In an embodiment, methods were investigated to determine the osteoinduction potential of a test material in an intermuscular implant site using the male athymic nude rat model. Eight athymic male rats were used in this study; one implant site per test material. The animals received two intermuscular implants between the adductor brevis and semimembranosus muscle groups. The animals were anesthetized and prepared for surgery. Pockets were created using sharp and blunt dissection in the muscle. After the incision over the implant site was made, the sample was placed into the muscle pocket, and then the pocket and skin were sutured closed. The animals were in-life for 28 days and observed daily for abnormal general health status.

At the end of the study duration, the animals were sacrificed, and the implants were removed. The tissues were fixed in 10% neutral buffered formalin prior to routine decalcification and processed into paraffin blocks. At least four sections were taken from each implant site, mounted on slides and stained with hematoxylin and eosin (H&E). Slides were viewed under a microscope and interpreted by a pathologist; the histopathology is semi-quantitative. A score was assigned to each implant site as either positive or negative for evidence of new bone formation elements.

In an embodiment, the animals used for the data disclosed herein were rats (Rattus norvegicus), athymic nude (RNU/RNU). The animals used were male. The animals weighed between 298.5-371.8 grams at the start of the study. The animals were approximately 11-13 weeks of age at the time of implants. In an embodiment, eight animals were used for the experiments disclosed herein. The animals were acclimated for a minimum of five days under the same conditions as the actual study.

Preparation of Samples

Samples that were wet were mixed to provide a homogenous sample. Samples that were dry were rehydrated with sterile saline. Samples were then loaded into sterile syringes. Syringes were front and back loaded with plungers to ensure the sample did not dehydrate prior to implantation. One syringe was made per implant site. Approximately 250±25 mg of test material was implanted into each site.

Histopathology Analysis

Explanted samples were decalcified and processed into paraffin. Sections nominally 3-6 microns thick were cut, mounted onto glass slides and stained with hematoxylin and eosin. Blocks were faced as necessary. At least four sections were taken from each implant site. A pathology report was generated which scored each individual implant site as either positive or negative for evidence of new bone formation elements; refer to Table 3. The field is defined as the entire implant material.

TABLE 3
Microscopic Evaluation of Osteoinduction
per Implant Site Semi-Quantitative Analysis.

| Grade | Estimated Cross-Sectional Area |
|---|---|
| NA | Not Applicable; No implant present |
| 0 | No evidence of new bone formation |
| 1 | Greater than 0% up to 25% of |
| | field shows evidence of new bone formation |
| 2 | 26-50% of field shows evidence of new bone formation |
| 3 | 51-75% of field shows evidence of new bone formation |
| 4 | 76-100% of field shows evidence of new bone formation |

Final determination of test material osteoinduction potential as either positive or negative was based on the histopathology analysis of the implant sites. The pathology report includes a summary of the evidence found in each implant site and the score given to each site.

Implants displaying a score of NA are considered either a complete resorption of test material or that no implant was detected upon explant. Implants displaying scores of 0 are considered non-osteoinductive. Implants displaying a maximum score of 1, 2, 3, and 4 are considered osteoinductive.

Assay validity was based on the successful implantation of the test material, implant sites grossly free from bacterial contamination, and microscopic evidence of the original test material present at the site. Assay validity will be based on the criteria above as well as scientific judgment.

Clinical Observations

All animals, except #3, gained or maintained a similar weight over the course of the study. Animal #3 lost weight, but only a 20% weight loss is considered significant; refer to Table 6. On Day 1 post implant, animal #2 was found with both incision sites open. They were closed with surgical glue and some scabbing was noted on both incision sites on Day 2 and Day 3. On Day 1 post implant, animal #3 was found with an approximate 4.5 cm×1.5 cm opening on the right incision site. The animal was anesthetized so the incision site could be cleaned, debrided, and closed with suture/surgical glue. Scabbing was noted until Day 5, when the animal was placed on treatment due to the scab appearing red and moist. The animal was on treatment through Day 13. No other abnormal clinical signs were noted for any of the animals during the course of the study.

Macroscopic Observations

All implant sites were positively identified and harvested. Macroscopic explant observations are detailed at least in Tables 4, 5, and 6.

Histopathology

Elements of new bone formation were observed in 9 out of 16 implant sites. Re-cuts of the blocks for the following lots were requested by the Sponsor: TL-001, TL-002, TL-005, TL-006, TL-013, and TL-14. Refer to Tables 5-7 for histopathological evaluation of each implant site.

The animals survived to the scheduled study endpoint. Samples TL-001, TL-009, TL-010, TL-011, TL-012, TL-013, TL-014, TL-015, and TL-016 met the histological criteria for evidence of osteoinduction, thereby demonstrating osteoinduction potential in the intermuscular implant site using the male athymic nude rat model. Samples TL-002, TL-003, TL-004, TL-005, TL-006, TL-007, and TL-008 did not meet the histological criteria for evidence of osteoinduction. Table 4 summarizes the in vivo osteoinduction assay for each sample.

TABLE 4
Summary of In Vivo Osteoinduction Assay

| | Validity Criteria | Interpretation of |
|---|---|---|
| Lot # | Met | Results |
| TL-001 | Yes | Osteoinductive |
| TL-002 | Yes | Non-Osteoinductive |
| TL-003 | Yes | Non-Osteoinductive |
| TL-004 | Yes | Non-Osteoinductive |
| TL-005 | Yes | Non-Osteoinductive |
| TL-006 | Yes | Non-Osteoinductive |
| TL-007 | Yes | Non-Osteoinductive |
| TL-008 | Yes | Non-Osteoinductive |
| TL-009 | Yes | Osteoinductive |
| TL-010 | Yes | Osteoinductive |
| TL-011 | Yes | Osteoinductive |
| TL-012 | Yes | Osteoinductive |
| TL-013 | Yes | Osteoinductive |
| TL-014 | Yes | Osteoinductive |
| TL-015 | Yes | Osteoinductive |
| TL-016 | Yes | Osteoinductive |

Summaries, Animal Data, Explant Observations, and Pathology Reports

(X) is the presence of element and (−) is the element is not present, and LL is left leg, RL is right leg.

TABLE 5
Summary of Pathology Report

| | | Observed Elements of New Bone Formation | | | | | | |
|---|---|---|---|---|---|---|---|---|
| Animal | | Chondroblasts/ | Osteoblasts/ | Cartilage/ | New | Bone | Original | Grade |
| Number | Site | Chondrocytes | Osteocytes | Osteoid | Bone | Marrow | DBM | (0-4) |
| 1 | LL | X | — | X | — | — | X | 1 |
| 2 | LL | — | — | — | — | — | — | 0 |
| 3 | LL | — | — | — | — | — | X | 0 |
| 4 | LL | — | — | — | — | — | — | 0 |
| 5 | LL | — | X | — | X | -— | X | 1 |
| 6 | LL | X | X | X | X | X | X | 2 |
| 7 | LL | — | X | — | X | — | X | 1 |
| 8 | LL | — | X | — | X | X | X | 1 |
| 1 | RL | — | — | — | — | — | X | 0 |
| 2 | RL | — | — | — | — | — | — | 0 |
| 3 | RL | — | — | — | — | — | X | 0 |
| 4 | RL | — | — | — | — | — | — | 0 |
| 5 | RL | X | X | X | X | — | X | 1 |
| 6 | RL | X | X | X | X | X | X | 2 |
| 7 | RL | X | X | X | X | X | X | 1 |
| 8 | RL | X | X | X | X | X | X | 1 |

TABLE 6
Animal Data

| | Implanted Lot Number | | Initial | Terminal | Weight |
|---|---|---|---|---|---|
| Animal | Left | Right | Weight | Weight | Change |
| Number | Site | Site | (g) | (g) | (g) |
| 1 | TL-001 | TL-002 | 301.1 | 317.9 | 16.8 |
| 2 | TL-003 | TL-004 | 320.2 | 333.0 | 12.8 |
| 3 | TL-005 | TL-006 | 371.2 | 327.1 | −44.1 |
| 4 | TL-007 | TL-008 | 310.5 | 327.2 | 16.7 |
| 5 | TL-009 | TL-010 | 371.8 | 368.8 | −3.0 |
| 6 | TL-011 | TL-012 | 319.8 | 343.2 | 23.4 |
| 7 | TL-013 | TL-014 | 345.7 | 363.6 | 17.9 |
| 8 | TL-015 | TL-016 | 298.5 | 322.9 | 24.4 |

TABLE 7
Macroscopic Explant Observations where IM is found
in or between muscle groups and where M is multiple
pieces scattered and where I is intact in a single piece.

| Animal | Left Site | | Right Site | |
|---|---|---|---|---|
| Number | Location | Scope | Location | Scope |
| 1 | IM | I | IM | M |
| 2 | IM | I | IM | I |
| 3 | IM | I | IM | I |
| 4 | IM | M | IM | I |
| 5 | IM | I | IM | M |
| 6 | IM | M | IM | I |
| 7 | IM | M | IM | I |
| 8 | IM | I | IM | I |

Histological analysis of all explants were performed upon animal sacrifice. All explants were evaluated using the scoring scale as summarized in Table 3.

The athymic rat osteoinductivity model will followed the method as described in ASTM Standard F04.4-F2529-13: Osteoinductivity in Mice or Rats. The cellulose nanofiber source was the National Renewable Energy Laboratory (NREL). In some embodiments, the sodium alginate (ALG) used was a 6.5% working stock concentration. In an embodiment, a 10 mM CaCl solution was used. In an embodiment, the demineralized bone matrix (DBM) used came from Australian Biotechnologies (ABT). In an embodiment, a negative control used non-inductive DBM that came from ABT and was inactivated using a guanidine extraction method.

In another embodiment, formulations used include the following: 5.1. F #1 Control (F1C): 6.0% CNF, 1.69% ALG and 30 second CaCl soak—NO DBM. 5.2. F #1 Active (F1A): Formula of F1C with 10% wt/wt DBM. 5.3. F #2 Control (F2C): 6.0% CNF, 2.99% ALG and 30 second CaCl soak—NO DBM. 5.4. F #2 Active (F2A): Formula of F2C with 30% wt/wt DBM. 5.5. F #3 Control (F3C): 6.0% CNF, 4.94% ALG and 2 min CaCl soak—NO DBM. 5.6. F #3 Active (F3A): 4.6% CNF, 3.8% ALG and 5 min CaCl soak with 23.1% wt/wt DBM. In an embodiment, sixteen 50 ml sterile centrifuge tubes were used to collect samples.

In an embodiment, an animal model was used that included non-GLP, athymic nude rat (Rattus norvegicus) with intermuscular pouch (between muscles) injections. In an embodiment, the sex of the rats was male and they had a weight of from about 298.5-371.8 g at the start of the investigation with an age from 11-13 weeks at the start of the investigation. All animals were acclimated for a minimum of five days under the same conditions as the actual investigation. A total of eight animals were used for this investigation. In an embodiment, the sample size was 250±25 mg per implant. In an embodiment, samples were sterilized on 26 Dec. 2017 by low dose e-beam (14.0-15.9 kGy).

All control and test groups were evaluated for intra-assay variability in duplicate in the same animal (2 implants per rat). In an embodiment, controls (N=4) were two DBM positive and two DBM negative controls experiments. In an embodiment, test articles (N=12) were three different formulations of the cellulose nanofibers all tested in duplicate with DBM (n=6−ACTIVE−“A”) and without DBM (n=6−CONTROL−“C”). The testing constants were a total sample mass of 5 g except for F3A, which had a final mass of 6.5 g. CNF concentrations used were 6% wt/wt, except for F3A, which had 3.8% wt/wt due to a higher alginate concentration. Testing variables included varying alginate concentrations including F1: 1.69%; F2: 2.99%; F3C: 4.94%; F3A: 3.80%; F1A: 10%; F2A: 30%; and F3A: 23%. In an embodiment, the implant date was 29 Dec. 2017 and an explant date was 26 Jan. 2018.

All animals survived to the scheduled necropsy date. Of the sixteen samples, ten of them were control samples, from five different formulations (all formulations were tested in duplicate). Each formulation had its own internal control to evaluate the CNF formulation performance without an active DBM component. Also included were controls to evaluate the performance of the DBM lot (as the active agent); a positive control (DBM powder no CNF) and a negative control (guanidine extracted DBM powder from same lot). All samples from each formulation group were implanted in the same animal.

Of the ten control samples, at least two samples yielded unexpected results; Sample 001 (DBM Negative Control) and Sample 014 (CNF-F3-CONTROL). These two samples were scored as OI positive (refer to Table 8) when neither sample contained an active osteoinductive agent. Without being bound by theory, possible explanations for the positive OI result one of the two DBM negative control samples are that: 1). the guanidine extraction method was not robust or reproducible enough, 2). The guanidine concentration (100 mM) may not have been high enough, 3). The temperature was not high enough or 4). The guanidine exposure was too short (45 min) to fully inactivate the bone morphogenic proteins. The presence of osteoinductive elements in the CNF control sample was unexpected, especially in light of the previous two control CNF formulations (F1C and F2C) not showing any signs of osteoinductivity. Differences between the third formulation group (F3C) and the two previous CNF formulations were the alginate concentration and the calcium chloride soak time. The third CNF formulation contained the highest alginate concentration (4.94) and had the longest calcium chloride soak time (2 min) for all of the samples. Although the DBM concentrations were also varied between the formulations, the third CNF formulation had less DBM (23%) than the second CNF Formulation (30%), and thus it is not a factor in the positive OI score. Like the negative controls, the duplicate F3C results were split, suggesting that the process may need to be improved to ensure a robust and reproducible outcome. Thus, osteoinductivity scores can be enhanced by altering the alginate and/or calcium chloride soak times.

Both samples from the DBM positive control group (011 and 012) resulted in osteoinductivity scores of two, meaning that this lot of DBM showed evidence of new bone formation in at least 26-50% of the implant. All other formulation and DBM negative control samples (002, 003, 004, 007, 008 and 013) were reported as OI negative, which was the expected result. The higher OI scores for the DBM positive controls over the other formulations suggest that the carrier material may be delaying osteogenesis in this short (28 day) in-vivo model and may not be reflective of the full remodeling or resorption capabilities of this biomaterial. The DBM is layered throughout the implant as compared to the powder where the powder is exposed unencumbered to the in-vivo model. This layered effect may prove to beneficial in more complex and higher animal models where the healing period extends out several months. Future investigations should include longer in-vivo periods (e.g. 3, 6, 9 or 12 months) looking for complete fusion or non-union endpoints to fully assess the capabilities of these new biomaterial formulations in clinical significant bone defects.

TABLE 8
Control Formulation Osteoinductivity Summary:

| | | OI | |
|---|---|---|---|
| | WuXi Apptec | Scores | |
| | BLINDED | Original | EXPECTED |
| Test Article | Sample ID | Score | RESULT |
| DBM NEGATIVE | 001 | 1 | NO |
| Control | 002 | 0 | YES |
| CNF-F1-CONTROL | 003 | 0 | YES |
| | 004 | 0 | YES |
| CNF-F2-CONTROL | 007 | 0 | YES |
| | 008 | 0 | YES |
| DBM POSITIVE Control | 011 | 2 | YES |
| | 012 | 2 | YES |
| CNF-F3 Control | 013 | 0 | YES |
| | 014 | 1 | NO |

Test Article Formulations:

Of the six-remaining active formulation test articles (refer to Table 9), there were at least two unexpected results. Samples 005 and 006, both from the CNF-F1-ACTIVE group were scored as non-inductive. All other samples (009, 010, 015 and 016) resulted in an OI score of 1 (>0-25% new bone formation). It is generally accepted within the industry that a minimum of 20% DBM must be used to yield a positive OI result Upon review, samples 005 and 006 only contained 10% due to the addition of alginate and therefore the zero scores were more indicative of a low DBM concentration than any negative interference from the CNF biomaterial.

TABLE 9
Active Formulation Osteoinductivity Summary

| | WuXi Apptec | OI Scores | |
|---|---|---|---|
| | BLINDED | Original | EXPECTED |
| Test Article | Sample ID | Score | RESULT |
| CNF-F1-ACTIVE | 005 | 0 | NO |
| (10% DBM) | 006 | 0 | NO |
| CNF-F2-ACTIVE | 009 | 1 | YES |
| (30% DBM) | 010 | 1 | YES |
| CNF-F3-ACTIVE | 015 | 1 | YES |
| (21% DBM) | 016 | 1 | YES |

As a result of these unexpected results, a re-cut was requested and performed on 13 Feb. 2018 in duplicate for each sample with discordant or unexpected results (i.e. samples 001, 002, 005, 006, 013 and 014). The re-cut process involved using the same histological block but fresh slices are taken deeper in the implant. If new bone elements are observed deeper within the implant that would confirm the original osteoinductive claim.

The re-cut results are presented in Table 10. Of the six samples that were re-evaluated, two sample scores (001 and 002) were reversed from the original score. Sample 001 (DBM negative control) was scored as zero (0) in the re-cut, which is consistent with the expected result of a guanidine inactivated DBM powder. Samples 005 and 006 (10% DBM concentrations) were confirmed to not have any osteoductive properties confirming the low DBM concentration. However, on samples 013 and 014, not only did the re-cut on sample 013 confirm the original osteoinductivity result but its duplicate sample 014 (CNF-F3-CONTROL) was also confirmed to be osteoinductive. This is a surprising result as the CNF biomaterial was thought to be inert yet both samples seem to validate the osteoinductive score with new bone elements deeper within the explant.

TABLE 10
Re-cut OI Sample Summary:

| | | OI Scores | | |
|---|---|---|---|---|
| | | | Re-Cut | EXPECTED |
| | BLINDED | Original Score | Score | RESULT |
| Test Article | Sample ID | (6 Feb. 2018) | (13 Feb. 2018) | (RE-CUT) |
| DBM | 001 | 1 | 0 | YES |
| NEGATIVE | 002 | 0 | 0 | YES |
| CONTROL | | | | |
| CNF-F1- | 005 | 0 | 0 | YES |
| ACTIVE | 006 | 0 | 0 | YES |
| CNF-F3- | 013 | 0 | 1 | NO |
| CONTROL | 014 | 1 | 1 | NO |

Based on the OI positive scores for both duplicate CNF-F3 control samples, a second re-cut was requested that involved scoring individual sections from each histopathology slide. This section grading report was performed on 1 Mar. 2018 for all samples from the third formulation (013, 014—CNF-F3 Controls and 015 and 016—CNF-F3 Active). The section grading report is a semi-quantitative method that can be used as an indicator on how pervasive new bone formation is throughout the entire implant (i.e. scoring a 1 or above on multiple sections vs. scoring a 1 or above on one or two sections of the same implant). In this step, another fresh slice is taken from the histology block and an osteoinductivity score for each section is reported. This is in contrast to the previous results, where only the highest score is reported from all of the sections (typically between 4 and 6 sections per sample).

The second re-cut section grades are presented in Table 11 below. Of the four samples, only one sample (013 CNF-F3 Control) resulted in a reversal of the original and re-cut #1 scores, where there were no visible signs of osteoinductivity on any of the six individual sections. The duplicate control sample (014 CNF-F3 Control) resulted in osteoinductivity grades of 1 in three out of the four sections (75%) suggesting that there were multiple points of new bone formation found throughout the control implant. In contrast, the active sample with DBM (016 CNF-F3 Active) only scored a 1 in 25% of the sections (1 out of four) and the duplicate active sample (015 CNF-F3 Active) had positive grade in two out of four (50%).

TABLE 11
Second Re-cut and Section Grading Summary

| | Blinded | Section | OI | Percent OI |
|---|---|---|---|---|
| Test Article | Sample ID | Number | Grade | Positive Sections |
| CNF-F3- | 013 | 1 | 0 | 0% |
| CONTROL | | 2 | 0 | |
| | | 3 | 0 | |
| | | 4 | 0 | |
| | | 5 | 0 | |
| | | 6 | 0 | |
| | 014 | 1 | 1 | 75% |
| | | 2 | 1 | |
| | | 3 | 0 | |
| | | 4 | 1 | |
| CNF-F3- | 015 | 1 | 1 | 50% |
| ACTIVE | | 2 | 0 | |
| | | 3 | 0 | |
| | | 4 | 1 | |
| | 016 | 1 | 0 | 25% |
| | | 2 | 0 | |
| | | 3 | 1 | |
| | | 4 | 0 | |

All results from all three evaluations (original, re-cut #1 and re-cut #2 section grading) are summarized in Table 12 below.

TABLE 12
Final Osteoinductivity Results Summary

| | | | Rat ID | OI Scores | | |
|---|---|---|---|---|---|---|
| | | | | | | Re-Cut |
| | BLINDED | # | (# of | | Re-Cut | #2 |
| Test Article | Sample ID | implants | Rats) | Original | #1 | (% Pos) |
| DBM | 001 | 1 | R-1 (1) | 1 | 0 | |
| NEGATIVE | 002 | 1 | | 0 | 0 | |
| Control | | | | | | |
| CNF-F1- | 003 | 1 | R-2 (1) | 0 | | |
| Control | 004 | 1 | | 0 | | |
| CNF-F1- | 005 | 1 | R-3 (1) | 0 | 0 | |
| ACTIVE | 006 | 1 | | 0 | 0 | |
| CNF-F2- | 007 | 1 | R-4 (1) | 0 | | |
| Control | 008 | 1 | | 0 | | |
| CNF-F2- | 009 | 1 | R-5 (1) | 1 | | |
| ACTIVE | 010 | 1 | | 1 | | |
| DBM | 011 | 1 | R-6 (1) | 2 | | |
| POSITIVE | 012 | 1 | | 2 | | |
| Control | | | | | | |
| CNF-F3 | 013 | 1 | R-7 (1) | 0 | 1 | 0 |
| Control | 014 | 1 | | 1 | 1 | 1 (75%) |
| CNF-F3- | 015 | 1 | R-8 (1) | 1 | | 1 (50%) |
| ACTIVE | 016 | 1 | | 1 | | 1 (25%) |
| TOTALS | | 16 | 8 | | | |

Positive DBM controls (Sample ID's 011 and 012) demonstrated a score of 1 or better on the in-vivo OI scoring scale. Both DBM positive controls resulted in OI scores of two, indicating the presence of between 26-50% new bone formation upon histological analysis. Negative DBM Controls (Sample ID's 001 and 002) demonstrate no evidence of new bone formation (OI Score of zero—0). The initial result for DBM control sample 001 was a positive OI score of 1, however upon the first re-cut there was no subsequent confirmation of new bone formation observed within the interior of the implant. All CNF controls and active implants did not result in the death of the animal and/or result in significant histological evidence of inflammation or immune reaction to the implant material. All animals survived to the expected necropsy endpoint and there was no histological evidence of an adverse reaction to the CNF biomaterial.

In an embodiment, CNF possesses osteoinductive or osteoconductive properties as a carrier material alone. In a split result after three rounds of histological analysis, the third CNF formulation indicated utility as an osteoinductive/osteoconductive carrier without the presence of DBM.

CNF+DBM product resulted in osteoinductivity within the in-vivo model (score of 1 or more). In all formulations except for the first formulation (CNF-F1), the carrier did not impede new bone formation with DBM levels greater than 23% (CNF-F2 and F3). Without being bound by theory, a reason the positive OI score was not observed for the first formulation may be that the DBM concentration was too low (about 10%).

The CNF biomaterial was evaluated in three different formulations where two variables were changed that were thought to be the most significant from an inductivity perspective; alginate concentration and DBM concentration. The first formulation (LOW) had the lowest concentration of alginate (1.69%) and demineralized bone matrix (10%). The second formulation (MID) increased alginate concentration to 2.99% and DBM concentration to a maximum of 30%. The third and final formulation (HIGH) sample group maxed out the alginate concentration (4.94%) without the addition of DBM whereas the active group contained a slightly lower alginate concentration (3.8%) due to the addition of the DBM (23.1%). The CNF concentration was kept constant at 6% in all formulations except for the third active formulation where the higher concentration of alginate made it very difficult to achieve a putty like consistency. In addition, a flash cross-linking step (30 seconds) was performed using a strong calcium chloride solution on the first two formulations to lock the DBM powder within the alginate/CNF matrix, thus creating a time release profile. The final formulation was soaked for a longer period of time (2 min) and had a noticeably crustier outer layer and firmer inner core as compared to the flash-soaked samples.

The cellulose nanofiber biomaterial was shown to be a novel carrier material when combined with demineralized bone matrix powder in excess of 10%. The only active cellulose nanofiber formulation that did not demonstrate a positive osteoinductivity score was the 10% DBM group. Without being bound by theory, it is thought that the DBM concentration was too low to yield any evidence of new bone formation. All other active formulations resulted in positive OI scores of 1, indicating the presence of new bone elements between 0 and 25%.

In an embodiment, an unexpected result occurred in the control group from the third CNF formulation that resulted in positive inductivity scores. This is not expected as the CNF biomaterial was thought to be inert. In an effort to confirm these initial results, all samples from the third formulation group were re-cut twice and in the second re-cut scored using a semi-quantitative histopathological evaluation. Semi-quantitative grading confirmed the initial positive OI results of both of the control samples.

A difference between the control groups in formulation three and the other two formulations was the extended calcium chloride soak time and the increased alginate concentration.

Although the handling characteristics were diminished because of the longer soak time, the presence of additional calcium solution that saturated the implant may have independently stimulated osteogenic cellular activity, thus resulting in the positive OI score deeper in the implant.

The CNF biomaterial demonstrated no evidence of biocompatibility issues and demonstrated positive osteoinductivity scores with the biomaterial alone without an active DBM group.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A bone repair composition comprising cellulose nanofiber useful as an osteoconductive matrix for cells to attach and adhere to during osteogenesis; and a derived inductive component, comprising allogenic cells and tissues added to stimulate and promote osteogenesis; and further comprising autologous derived cells and tissues; and further comprising recombinant proteins, cytokines, and growth factors; and further comprising compounds useful for bone remodeling comprising calcium, magnesium, and vitamin D; and further comprising compounds useful for the mitigation of infection comprising antibiotics and heavy metal ions.

2. The composition of claim 1, that is biocompatible, non-toxic, anti-inflammatory, non-immunogenic biomaterial derived from renewable sources.

3. The composition of claim 2 comprising wood, plants, trees, paper pulp, bacteria, and algae.

4. The composition of claim 1 further comprising cellulose nanofiber derived from plant-based material with a length of between 50 nanometers and 100 micrometers and diameter of 2 nanometers and 20 nanometers.

5. The composition of claim 1 further comprising cellulose nanofibers comprising fiber bundles with lengths of between about 50 nanometers and 1 millimeter and diameters of between about 2 nanometers and 3 micrometers.

6. The composition of claim 5 comprising wherein the fiber bundles have a water content of between about 50% to about 80%.

7. The composition of claim 5 comprising fiber bundles with an alginate concentration of between about 1% to about 10% by weight.

8. The composition of claim 5 further comprising fiber bundles and powder cut from cortical bone demineralized to a calcium content of no more than 10%.

9. The composition of claim 5 further comprising fiber bundles and bone chips and/or bone cubes cut from non-demineralized cancellous bone with sizes of between about 1 mm to about 15 mm.

10. The composition of claim 8 further comprising calcium chloride at a concentration from about 0.1 M to about 10 M wherein the solution is allowed to cross-link between about 0.1 min to about 60 min.

11. The composition of claim 8 further comprising less than 10% water by weight, less than 50% water by weight, less than 80% water by weight and greater than 80% water by weight.

12. The composition of claim 1, further comprising an excipient selected from the group consisting essentially of alginate, glycerol, lecithin, sodium carboxy methyl cellulose, hyaluronic acid and derivatized hyaluronic acid.

13. The composition of claim 1 further comprising a cross-linking agent selected from multivalent cations comprising calcium, magnesium, and further comprising counter ions including chlorides, sulfates, carbonates, and nitrates.

14. The composition of claim 1 further comprising vitamin A, vitamin B, vitamin C, vitamin D, vitamin E and vitamin K.

15. The composition of claim 1 further comprising a demineralized bone matrix component.

16. The composition of claim 1 further comprising a non-demineralized bone matrix component.

17. The composition of claim 1 further comprising growth factors, cytokines, antimicrobial agents, antifungal agents, bioglass and its derivatives, tri-calcium phosphates and its derivatives, chitosan and its derivatives, collagen and its derivatives.

18. A method for repairing bone using the composition of claim 13 wherein the cross-linking agent is added to the rest of the composition and wherein the reaction time of the cross-linking agent with the rest of the composition is directly proportional to resorption time in-vivo.

19. The method of claim 18, wherein the concentration of the cross-linking agent is proportional to resorption time in-vivo.

20. The method of claim 19 wherein the resorption time in-vivo is between 1 month to about 12 months.
